(19) United States
(12) Patent Application Publication
(10) Pub. No.: US 2014/0020130 A1
(43) Pub. Date: Jan. 16, 2014

(54) csRRM2 GENE AND ITS USE FOR IMPROVING TRAITS IN INDUSTRIAL CROPS

(71) Applicants: Fuguang Li, Anyang (CN); Chuanliang Liu, Anyang (CN); Zhixia Wu, Anyang (CN); Fenglian Li, Anyang (CN); Xueyan Zhang, Anyang (CN); Haihong Shang, Anyang (CN); Chaojun Zhang, Anyang (CN); Depei Kong, Anyang (CN); Qianhua Wang, Anyang (CN); Yufen Wang, Anyang (CN); Jinshui Yang, Shanghai (CN); Fan Sun, Shanghai (CN); Weiwei Qi, Shanghai (CN); Xiaoyin Qian, Shanghai (CN); Xiaojin Luo, Shanghai (CN)

(72) Inventors: Fuguang Li, Anyang (CN); Chuanliang Liu, Anyang (CN); Zhixia Wu, Anyang (CN); Fenglian Li, Anyang (CN); Xueyan Zhang, Anyang (CN); Haihong Shang, Anyang (CN); Chaojun Zhang, Anyang (CN); Depei Kong, Anyang (CN); Qianhua Wang, Anyang (CN); Yufen Wang, Anyang (CN); Jinshui Yang, Shanghai (CN); Fan Sun, Shanghai (CN); Weiwei Qi, Shanghai (CN); Xiaoyin Qian, Shanghai (CN); Xiaojin Luo, Shanghai (CN)

(73) Assignees: FUDAN UNIVERSITY, SHANGHAI (CN); COTTON RESEARCH INSTITUTE OF THE CHINESE ACADEMY O, ANYANG (CN)

(21) Appl. No.: 13/813,264

(22) PCT Filed: Nov. 4, 2010
(86) PCT No.: PCT/CN10/78434

§ 371 Date: Jan. 30, 2013

(51) Int. Cl. C12N 15/82; C07K 14/415

(52) U.S. Cl. CPC C12N 15/8261; C07K 14/415
USPC 800/270; 530/324; 536/23.6; 435/320.1; 435/419; 435/6.12; 800/298

(57) ABSTRACT

The present invention clones the second RNA recognition motif (RRM2) gene of FCA-γ gene in Brassica napus, and transforms the RRM2 domain gene into cotton by transgenic method. The transgenic line exhibits characteristics of yield increasing, fiber quality enhancement and senescence resistance. The results indicate that the RRM2 domain gene can be used for improving traits in industrial crops.

### CROSS-REFERENCE TO RELATED APPLICATIONS

This application is a U.S. National Phase Application of PCT International Application PCT/CN2010/078434, filed Nov. 4, 2010, the contents of which are incorporated herein by reference in their entireties for all purposes.

### TECHNICAL FIELD

The invention belongs to the field of plant genetic engineering, particularly relates to a RRM domain gene of Brassica napus FCA gene, and the use of this domain gene for improving economic traits in crops.

### BACKGROUND OF THE INVENTION

Currently, the methods for genetically modified crops by transgening are mainly based on the relationship between functional genes and phenotypic traits. By using constitutive expression vectors or tissue-specific expression vectors, the gene of interest can be introduced into target plant to improve specific economic traits or resistant traits of the crop. The genes for use are all derived from native genes. As the sequence and function of these genes must be known at first, their application is limited to a great extent. “The method for transgenically improving economic traits of a crop by using FCA gene RNA domain” (CN200410084319.3) discloses a method for genetically modified crops by using RNA domain of functional genes. This method has expanded the scope of the transgenic utilization of the functional genes, and can produce different effects from conventional transgenic methods.

Arabidopsis thaliana FCA (Flowering Control Activator, FCA) gene (Macknight, R. et. al., Cell, 1997. 89(5): 737-745.) has 20 introns, in which the alternative splicing of the third and the thirteenth introns results in 4 different mature transcripts, named as FCA-α, FCA-β, FCA-γ, and FCA-δ, respectively; wherein only the protein (747 aa) encoded by FCA-γ prompts blooming, which has 2 RRM (RNA recognition motif) domains and one WW domain; the protein encoded by FCA-δ only has two RRM domains without the WW domain. Rice FCA (Du, X., et al. (2006). DNA Sequence 17(1): 31-40) gene can be in 4 alternative splicing forms, named as OsFCA-1, OsFCA-2, OsFCA-3, and OsFCA-4, respectively. OsFCA-1 (738 aa) is homologous to Arabidopsis thaliana FCA-γ, and also has two RRM domains and one WW domain; OsFCA-2 is 102 bp less than OsFCA-1, and OsFCA-2 is absent of the WW domain.

FCA protein selectively uses different polyadenylation sites in FCA pre-mRNA to regulate its expression level of alternative splicing. The binding of the FCA's WW domain and FY protein promotes the use of proximal polyadenylation site, and thus contributes to generate the useless alternatively spliced transcripts of FCA. RRM domain of Rice FCA protein is highly conserved in plants, and has a very high degree of identity in Triticum aestivum (90%), Hordeum vulgare (90%), Lolium perenne (82%), Zea mays (81%), Ricinus communis (76%), Populus (70%), Vitis vinifera (68%), Arabidopsis thaliana (68%), Brassica napus (64%) and other plants.

In Brassica napus, FCA-γ(GenBank: AF414188.1) is homologous with Arabidopsis thaliana FCA-γ. The protein encoded by Brassica napus transcript FCA-γ(GenBank: AAL61622.1, 715 aa) has the same protein structure with that encoded by Arabidopsis thaliana FCA-γ, and also contains two RRM domains and one WW domain, wherein amino acids 103-166 constitute the first RRM domain and amino acids 194-270 constitute the second RRM domain, and amino acids 570-600 constitute the WW domain.

Upon searching, no reports have been found about using domain of Brassica napus FCA-γ to improve yield or quality traits of cotton.

### SUMMARY OF THE INVENTION

The object of the present invention is to provide a protein associated with crop yield or quality traits. This protein is derived from the second RRM domain of Brassica napus FCA.

Another object of the present invention is to provide a gene encoding the above protein, which can be used for transgenically improving the economic traits of crops, improving yield and quality of dicotyledonous plants.

The third object of the present invention is to provide a method for improving crop yield or quality traits by using the above gene.

The fourth object of the present invention is to provide a method for breeding cultivars by using transgenic plants with the above-mentioned gene.

The technical solutions of the present invention are as follows: A protein associated with crop yield or quality traits, which is named as Bn-csRRM2 (cell-size RNA recognition motif) and is shown as the following (a) or (b):

(a) a protein consisting of the amino acid sequence of SEQ ID No: 1;

(b) a protein consisting of the amino acid sequence of SEQ ID No: 1 with deletion, addition or substitution of one or more amino acid residues, and associated with crop yield or quality traits.

The “crop” mentioned above may be cotton (Gossypium hirusute L.), maize (Zea mays L.), rice (Oryza sativa L.), wheat (Triticum aestivum L.), barley (Hordeum L.), or rape (Brassica napus L.), etc.

The “crop yield or quality traits” mentioned above may be cotton single boll weight, bolls per plant or fiber quality, etc. Said cotton “fiber quality” traits refer to fiber length, fiber strength or fiber fineness, etc.

A gene encoding the protein associated with crop yield or quality traits which is shown as the following 1) or 2) or 3):

1) a gene consisting of the nucleotide sequence of SEQ ID No: 2;

2) a gene consisting of the nucleotide sequence of SEQ ID No: 2 with mutation, substitution or deletion of one or more bases, and encoding the protein associated with crop yield or quality traits.

3) a gene consisting of a nucleotide sequence which can hybridize with the DNA sequence defined by SEQ ID No.: 2 under stringent conditions and encoding the protein associated with crop yield or quality traits.

An expression vector comprising the above encoding gene is provided.

A transgenic plant cell line comprising the above encoding gene is provided.

A transgenic plant tissue comprising the above encoding gene is provided.

A transgenic plant comprising the above encoding gene is provided.

The “vector” in the above “expression vector” is PBI121 or pBin438; preferably pBin438.

A method is provided for improving the crop yield or quality traits comprising introducing the above gene associated with crop yield or quality traits into a host plant, so as to obtain a plant with improved yield or quality.

The “host plant” above refers to cotton, maize, rice, wheat, rape or barley, and among others.

Taking cotton as an example to illustrate the method for transgenic breeding cultivars by using the above gene associated with crop yield or quality traits, the method comprising the steps of:

(1) Bn-csRRM2 cloning: total RNA is extracted from rape, and then used as a template for reverse transcription to obtain cDNA; then the cDNA is used as a template for PCR amplification by using Bn-csRRM2-S and Bn-csRRM2-A as primers, the amplified product is tested in 1% agarose gel electrophoresis, and then the amplified fragments are recovered to obtain the Bn-csRRM2 gene fragment; said primers are:

Bn-csRRM2-S:
(SEQ ID No: 3)
5′- GAGGATCCATGGGTGCGGTAGAGTT -3′
Bn-csRRM2-A:
(SEQ ID No: 4)
5′- CGTAGATCTTGTGCCACTTCCCTTG -3′

(2) Construction of expression vector: Bn-csRRM2 gene fragments obtained from (1) are connected to the vector pGEM-T to get the intermediate vector pGEM-T-csRRM2; the intermediate vector pGEM-T-csRRM2 and pBIN438 are subjected to XbaI and BamHI double digestion, and digested fragments are recovered to obtain the Bn-csRRM2 fragment of interest and pBIN438 vector backbone; by using T4 DNA ligase, the Bn-csRRM2 is connected to the vector backbone pBIN438 to obtain the expression vector pBIN438-csRRM2, which is then transferred into Agrobacterium tumefaciens (such as strain LBA4404) to obtain strains comprising Bn-csRRM2 gene;

(3) Transformation: the cotton seeds are husked, inoculated on MS medium (regular), and then grown under light conditions for 48˜72 h. Shoot tips are stripped when the cotyledons slightly open: remove the cotyledons from seedling, leave 0.4˜1.0 cm hypocotyledon, remove the shoot tips under a dissecting microscope, and reserve two leaf primordia; the expression carrier pBIN438-csRRM2 is bombarded into the shoot apex by gene gun bombardment;

(4) Screening and obtaining the regeneration seedlings: the bombarded material is grown for 3˜7 d till the juvenile leafs become visibly green; gradient screening is carried out by sequentially using the kanamycin in concentration gradient of 65 mg/L, 80 mg/L, 90 mg/L to 100 mg/L, with an interval of 7˜15 d, to select kanamycin-resistant plants, and get the regeneration seedlings; the screening media used are respectively: MS medium without hormones; G1 medium: MS medium+2.0 mg/L KT+2.0 mg/L IAA; G2 medium: MS medium of +0.1 mg/L IAA+0.1 mg/L 2,4-D;

(5) Grafting and transplanting: cotton seedling stems grown on a nutrient bowl and 3 to 4 days after seedling emergence are used as the rootstock, the regeneration seedlings obtained from the step (4) are grafted and planted in a greenhouse, and PCR amplification identification and southern blotting detection are performed on the successfully grafted plants, plants identified as comprising Bn-csRRM2 gene are selected, strictly inbred, and cottons transferred with the Bn-csRRM2 gene are then obtained.

The method for cultivating plant varieties by using a transgenic plant with the encoding gene, comprising using the transgenic plant with the encoding gene as one of the parents, and cultivating plant varieties with improved yield or quality traits by backcross or hybrid methods.

Said plant is cotton, maize, wheat, barley, rape, rice, etc.

A hybrid breeding method which uses the above transgenic plant as one of the parents to obtain the hybrid.

A PCR kit for detecting the above encoding gene, in which the primers used for the PCR amplification are:

(SEQ ID No: 7)
5′ AGTCGTGGATGCGGGTTTGTTA 3′
(SEQ ID No: 8)
5′ GCAAGGCGATTAAGTTGGGTAA 3′.

Compared with the prior art, the present invention has advantages and beneficial effects: taking cotton as an example, compared with non-transgenic controls, the cotton with csRRM2 gene of the present invention has the following characteristics. (1) Cotyledons, leaves, flowers, bracts, petioles, plant heights, bolls, among others are enlarged. (2) Single boll weight increases from 4.8-5.2 g of subject to 6.4-7.8 g; bolling increases, boll number per plant increases from 13.5 to 17.7; and yield per plant increases from 15 to 50%. (3) The fiber quality of transgenic material is improved, and compared with the transgenic subject, the fiber length increases by approximately 12%, the breaking strength increases. (4) Pollens are enlarged. (5) Cells are enlarged. (6) Transgenic cotton is resistant to senescence.

### BRIEF DESCRIPTION OF THE DRAWINGS

FIG. 1. Electrophoresis profile of csRRM2 gene product by PCR amplification; where lane 1 is the Marker: lane 2 is PCR Product: about 300 bp; the band of about 100 bp is a primer dimer.

FIG. 2. The structural profile of expression vector pBIN438-csRRM2.

FIG. 3. Electrophoresis profile of expression vector detection; where lane 1 is Marker; lane 2 is pBIN438 plasmid.

FIG. 4. Comparison of cotton cotyledons, wherein 1 is CCRI 12; 2 is the transgenic CCRI 12.

FIG. 5. Comparison of cotton bract, wherein 1 is CCRI 12; 2 is the transgenic CCRI 12.

FIG. 6. Comparison of cotton leaves, wherein 1 is CCRI 12; 2 is the transgenic CCRI 12.

FIG. 7. Comparison of cotton flowers, wherein 1 is CCRI 12; 2 is the transgenic CCRI 12.

FIG. 8. Comparison of bolls, wherein 1 is CCRI 12; 2 is the transgenic CCRI 12.

FIG. 9. Comparison of cotton ovary, wherein 1 is CCRI 12; 2 is the transgenic CCRI 12.

FIG. 10. Comparison of cotton flap, wherein 1 is CCRI 12; 2 is the transgenic CCRI 12.

FIG. 11. Comparison of boll shell, wherein 1 is CCRI 12; 2 is the transgenic CCRI 12.

FIG. 12. Comparison of cotton fiber, wherein 1 is CCRI 12; 2 is the transgenic CCRI 12.

FIG. 13. Comparison of cotton plants, wherein 1 is CCRI 12; 2 is the transgenic CCRI 12.

FIG. 14. Comparison of cotton field growth, wherein 1 is CCRI 12; 2 is the transgenic CCRI 12.

### DETAILED DESCRIPTION OF THE INVENTION

The present invention will be further described below in conjunction with the embodiments, but the scope of protection of the present invention is not intended to be limited. The following embodiments, if not particularly indicated, all use the conventional methods. The reagents used are commercially available.

### Example 1

### Cloning of Rape Bn-csRRM2 Gene

1. Obtainment and Purification of Rape cDNA

(1) Extraction and Purification of Rape Total RNA:

(a) 0.5 g cryopreserved rape leaf samples were ground into powders in liquid nitrogen, and RNAiso Plus (TaKaRa Code: D9108A) was added to completely cover the sample. The sample was then allowed to stand at room temperature till completely thawed. The sample was ground continuously till the lysate of the sample was transparent, to obtain a homogenate solution.

(b) The homogenate solution stood at room temperature for 5 minutes; then was subjected to centrifugation at 12,000 g for 5 minutes at 4° C. The supernatant was taken, and added with chloroform (⅕ of RNAiso Plus in volume). Centrifugation tube cap was closed tightly. The tube was shaken vigorously by hand for 15 seconds, until the solution was emulsified sufficiently. Then the solution was allowed to stand at room temperature for 5 minutes, and subjected to centrifugation at 12,000 g for 15 minutes at 4° C. The supernatant was pipetted, the white protein layer in the middle and the organic phase with color in the lower layer were discarded. The supernatant was added with an equal volume of isopropanol, mixed, and allowed to stand at 15˜30° C. for 10 minutes, and then subjected to centrifugation at 12,000 g for 10 min at 4° C. The supernatant was discarded, 1 ml of 75% ethanol was added and subjected to centrifugation at 12,000 g for 5 minutes at 4° C. The supernatant was discarded. The resulted precipitate was rape total RNA, which was dried at room temperature for 2 to 5 minutes. RNase free ddH₂O was added to dissolve the precipitate such that the RNA precipitate was completely dissolved.

(c) The reaction solution was formulated as follows: 20˜50 μg total RNA from (b), 5 μl 10× DNase I Buffer, 20 U RNAase inhibitor, 2 μl (10 U) DNase I (RNase-free), adjusted by RNase Free ddH₂O to a total volume of 500. The reaction solution stood at 37° C. for 20 min, and was adjusted by 500 RNase Free ddH₂O to a total volume of 1000. An equal volume of phenol/chloroform/isoamyl alcohol (25:24:1) was added and mixed together, and then kept at room temperature, subjected to centrifugation at 13,500 rpm for 5 min. The supernatant was taken, added with an equal volume of chloroform/isoamyl alcohol (24:1), mixed, and subjected to centrifugation at 13,500 rpm for 5 min at room temperature. The supernatant was extracted, added with 10 μl 3M sodium acetate and 2500 cold ethanol, kept on ice for 10 min, subjected to centrifugation at 13,500 rpm for 15 min at 4° C., and the supernatant was discarded. The precipitate was washed with 500 μl of 70% cold ethanol, subjected to centrifugation at 13,500 rpm for 5 min at 4° C., and the supernatant was discarded. The precipitate was dried and dissolved with 50 μl RNase free ddH₂O, and stored at −80° C.

The absorbance of samples at 260 nm and 280 nm was measured with a spectrophotometer. The A260/A280 value of the resulted sample was 2.05, which indicated the resulted RNA was not degraded and the purity met the requirement.

(2) Obtainment of Rape cDNA

The RT reaction solution was prepared with the following components (the reaction solution was formulated in accordance with the product specification).

PrimeScript® RT reagent Kit (TaKaRa Code: DRR037A)

RT reaction solution: 2 μl 5× PrimeScript® Buffer, 0.50 PrimeScript® RT Enzyme Mix I, 0.5 μl Oligo-dT Primer (50 μM), 0.50 Random 6 mers (100 μM), 500 ng RNA, adjusted by RNase Free dH₂O to a total volume of 10 μl.

Reverse transcription reaction conditions: 37° C. for 15 min, 85° C. for 5 sec to get cDNA.

(3) PCR Amplification of Bn-csRRM2

The primers were designed according to the full-length cDNA sequence of rape (Brassica napus napus L.) FCA-γ (GenBank: AF414188.1), and recognition sites of restriction enzymes BamHI and XbaI and protecting bases were introduced at both ends of the primers. The primers' sequences are as follows:

Bn-csRRM2-S:
(SEQ ID No: 3)
5′-GAGGATCCATGGGTGCGGTAGAGTT-3′
Bn-csRRM2-A:
(SEQ ID No: 4)
5′-CGTAGATCTTGTGCCACTTCCCTTG-3′

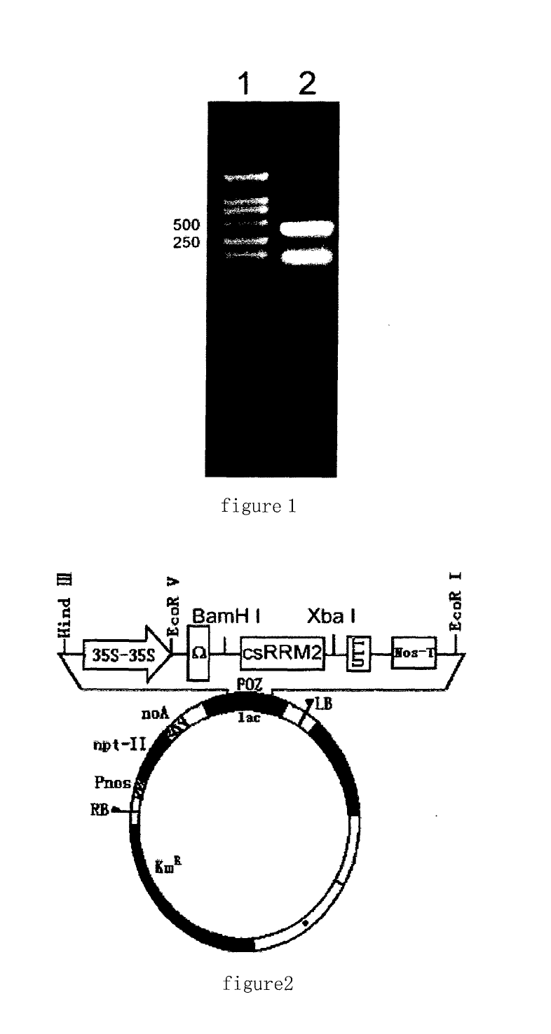

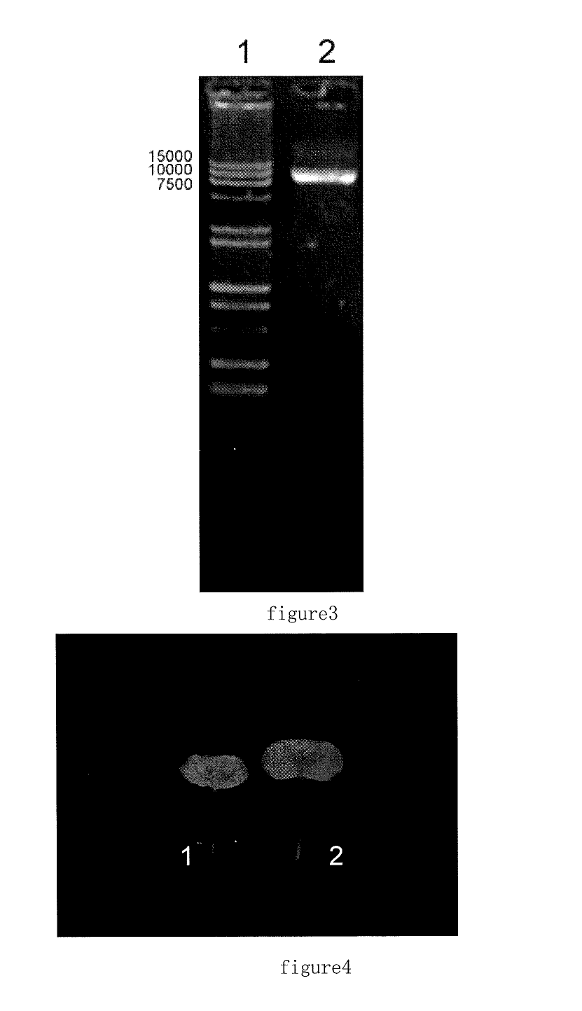

Amplification was performed by using cDNA from (2) as the template, Bn-csRRM2-S and Bn-csRRM2-A as the primers. The PCR reaction system was: 0.50 Takara LATaq (5 U/μl), 5.00 μl 10×LA PCR Buffer II (Mg2+ Free), 5.0 μl MgCl₂ (25 mM), 8.0 μl dNTP Mixture (each 2.5 mM), 1.0 μl forward primer Bn-RRM2-S (10 mM), 1.0 μl reverse primer Bn-RRM2-A (10 mM), 0.5 μl cDNA template, 29 μl sterilized dH₂O. The PCR reaction condition was as follows: 95° C. 2 min; 94° C. 30 s, 60° C. 30 s, 72° C. 1 mM, 30 cycles; 72° C. 10 mM. The resulted PCR product was subjected to 1% agarose gel electrophoresis detection. The results (FIG. 1) showed a specific band at about 300 bp upon amplification, another band was primer dimer. The obtained specific band was recovered and sequenced by Shanghai Yingjun Biotechnology Co. Ltd. The sequence is shown as SEQ ID NO: 2 and named as Bn-csRRM2.

### Example 2

### Construction of csRRM2 Gene Expression Vector

(1) Recovery of PCR amplification product: the band at about 300 bp in the PCR amplification product in Example 1 was recovered by AxyPrep™ DNA Gel Extraction Kit (Cat No. AP-GX-250, AXYGEN Co.) According to the manual thereof.

(2) Ligation of the fragment of interest to pGEM-T vector (Promega)

The ligation was carried out by using pGEM®-T Easy Vector System I (Cat. # A1360, Promega, Madison, Wis., USA) according to the manual thereof.

pGEM-T vector was instantaneously centrifuged to the bottom of the tube. 2× Rapid Ligation buffer was volute oscillated, and the reaction system was formulated in a 0.2 ml centrifuge tube: 50 μl 2× Rapid Ligation buffer, 10 pGEM-T Easy Vector (50 ng), 1 μl T4 DNA ligase (3 u/ml), 30 PCR product (Bn-csRRM2) (25 ug/ml). The reaction system was pipetted up and down to mix, kept at 4° C. overnight, so as to obtain a plasmid pGEM-T-csRRM2.

(3) Transformation of Plasmid pGEM-T csRRM2

50 μl cryopreserved Escherichia coli DH5a competent cells (prepared according to conventional methods) were dissolved in a centrifuge tube at 4° C.; 10 μl pGEM-T-csRRM2 obtained in step (2) was added to each centrifuge tube, gently mixed, and placed on ice for 30 min; and then the centrifuge tube was placed in a thermostatic water bath of 42° C. for 90 s without shaking; the centrifuge tube was quickly transferred to an ice bath to be cooled for 2 min. Each centrifuge tube was added with 2000 liquid LB (without antibiotics), then kept 180 rpm in a water bath at 37° C. for 50 min. Each LB ampicillin plate was plated with 16 μl X-gal (5-bromo-4-chloro-3-indole glycoside) and 4 μl IPTG (isopropyl thio-β-D galacto-glucoside), and allowed to stand for 30 min. All of the liquid in the centrifuge tube was smeared on an LB ampicillin plate, and allowed to stand for 30 min. The plate was inverted, and incubated overnight at 37° C. for 12˜16 h. The plate was placed at 4° C. for 1 hour to let the blue color fully developed. 10 white colonies were selected to inoculate in 600 μl centrifuge tubes with liquid LB (containing ampicillin, 100 mg/L), respectively, and then shaken overnight. The positive clone (pGEM-T-csRRM2) was verified by sequencing with T7 and SP6 primers. Simultaneously the ligation product without the DNA of interest was used as transformation control. Sequencing results showed that cloned Bn-csRRM2 gene fragment was correct, without mutation.

(4) Preparation and Purification of Plasmid DNA

1-4 ml bacteria solution cultured in LB medium overnight from step (3) was subjected to centrifugation at 12,000 g for 1 min, and the supernatant was discarded. The bacterial precipitate was suspended in 2500 Buffer SI added with RNase A (kit: AxyPrep™ Plasmid Miniprep Kit (Cat. AP-MN-P-250), hereinafter was the same). The bacterial precipitate was suspended homogenously, without leaving any small bacteria block. 250 μl Buffer S2 was added and mixed upside down homogenously for 4-6 times to get a transparent solution. 3500 Buffer S3 was added and mixed upside down homogenously for 6-8 times, subjected to centrifugation at 12,000 g for 10 min. The supernatant was transferred to a DNA preparation tube (placed in a 2 ml centrifuge tube) and subjected to centrifugation at 12,000 g for 1 min. The preparation tube was placed back to the centrifuge tube, added with 5000 Buffer W1, centrifuged at 12,000 g for 1 min, and the filtrate was discarded. The preparation tube was placed back to the centrifuge tube, added with 7000 Buffer W2, centrifuged at 12,000 g for 1 min, and the filtrate was discarded. Washing was performed again with 7000 Buffer W2 in the same way, and the filtrate was discarded. The preparation tube was placed back to a 2 ml centrifuge tube, centrifuged at 12,000×g for 1 min. The preparation tube was transferred into a new 1.5 ml centrifuge tube, 600 water was added onto the center of the DNA preparation membrane, and allowed to stand at room temperature for 1 min, then subjected to 12,000 g centrifugation for 1 min. 50 plasmid DNA was used for 1% agarose gel electrophoresis.

The results (FIG. 2) show that the plasmid extracted from pBIN438 empty vector is 13 Kb in size, the plasmid extracted from pGEM-T-csRRM2 is approximately 3.4 kb in size.

(5) Digesting the Fragment of Interest of Plasmid DNA

The plasmid DNA was digested with a restriction enzyme (NEW England Biolabs). The digestion reaction was as follows: 1 μg DNA, 2 μl 10× digestion buffer, 0.5 μl XbaI, 0.5 μl BamHI, supplied with ddH₂O to 20 μl. The reaction was carried out at 37° C. for 4 hours and then the digested plasmid DNA was subjected to 1% agarose gel electrophoresis. pBIN438 empty vector digested with XbaI and BamHI displayed a band of about 13 kb in electrophoresis. pGEM-T-csRRM2 digested with XbaI and BamHI displayed a fragment of about 3 kb and a fragment of about 300 bp in electrophoresis.

(6) Recovery and Ligation of the Fragment of Interest

The fragment was recovered by using AxyPrep™ DNA Gel Extraction Kit (Cat No. AP-GX-250) according to the manual. The fragment Bn-csRRM2 of interest was ligated to an expression vector pBIN438 by using T4 DNA ligase (NEW ENGLAND Biolabs). The ligation reaction system was as follows: 50 ng vector DNA, 200 ng exogenous fragment, 2 μl 10× ligation buffer, supplied with ddH₂O to 20 μl, and 1 μl T4 DNA ligase. The obtained plasmid was name as pBIN438-csRRM2.

The ligation product was used to transform E. coli DH5a competent cells. Single colonies were picked up and positive clones containing a recombinant plasmid were verified by PCR using specific primers Bn-RRM2-S and Bn-RRM2-A for PCR and two-direction sequencing.

Result: the open reading frame (the open reading frame of Bn-csRRM2 gene in well constructed vector pBIN438-csRRM2) in the sequence after matching the two-direction sequencing results was shown in SEQ ID No.: 5, which encodes the amino acid sequence of SEQ ID No: 6.

The structure of obtained expression vector pBIN438-csRRM2 is shown in FIG. 2.

### Example 3

### Obtaining the Bn-csRRM2 Gene Transgenic Cotton

1. The preparation of the stock solutions of the MS medium

Stock solution 1: KNO₃ 19,000 mg/L, NH₄NO₃ 16,500 mg/L, MgSO₄.7H₂O 370 mg/L, KH₂PO₄ 1700 mg/L, and CaCL₂.2H₂O 4400 mg/L were dissolved in 1000 ml of distilled water, and mixed uniformly.

Stock solution 2: MnSO₄.H₂O 1690 mg/L, ZnSO₄.7H₂O 860 mg/L, H₃BO₃ 620 mg/L, KI 83 mg/L, Na₂MOO.5H₂O 25 mg/L, CuSO₄.5H₂O 2.5 mg/L, and CoCl₂.6H₂O 2.5 mg/L were dissolved in 1000 ml of distilled water, and mixed uniformly.

Stock solution 3: glycine 200 mg/L, thiamine hydrochloride 40 mg/L, pyridoxine hydrochloride zinc 50 mg/L, nicotinic acid 50 mg/L, and inositol 10,000 mg/L were dissolved in 1000 ml of distilled water, and mixed uniformly.

Stock solution 4: Naₑ-EDTA 7.45 g/L, FeSO₄.7H₂O 5.57 g/L were dissolved in 1000 ml of distilled water, and mixed uniformly.

MS medium preparation: 100 ml stock solution 1, 10 ml stock solution 2, 10 ml stock solution 3, 5 ml stock solution 4, and 30 g sucrose were dissolved in a small amount of distilled water, to 990 ml by adding water, pH adjusted to 5.8, and then 0.2 g GEL Rite gellan gum was added and mixed, to a volume of 1 L.

2. Bn-csRRM2 Gene Transgenic Cotton was Obtained in Accordance with the Following Method:

(1) The cotton CCRI 12 was used as a transgenic subject. The seeds removed husks, and inoculated on MS medium (composition of the medium see above) for culture at 28° C. in light condition for 48˜72 h.

(2) The shoot tip was peeled when the cotyledons slightly opened: cotyledons were removed from the seedling, 0.4˜1.0 cm hypocotyls axis was retained, and the shoot tips were stripped out under dissecting microscope, and two leaf primordia were left. The shoot tips were bombarded with extracted gene plasmid DNA by using gene gun bombardment. And then un-bombarded material was used as the control.

(3) The bombarded material firstly was recovery cultured until the young leaves became visibly green, typically 3 to 7 days;

(4) After recovery culture, screening culture was started using kanamycin (Sigma) as a screening agent. With 65, 80, 90, 100 mg kanamycin L-1 and above, at intervals of 7˜15 d, the transgenic regeneration plants were gradually screened out. The screening media used were respectively: MS medium without hormones; G1 medium: MS medium+2.0 mg • L-1 KT+2.0 mg • L-1 IAA; G2 medium: MS medium+0.1 mg • L-1 IAA+0.1 mg • L-1 2, 4-D.

(5) Transplantation of the regenerated resistant plants: resistant plants were transplanted to the greenhouse by grafting. The grafting was as follows: the cotton seeds were planted in nutrient bowls, wetted from the bowl bottom up forward slowly by watering under the temperature of 28-30° C., and the seedling emergence time was about 3-4 days. Well grown cotton rootstocks seedlings were gently split with a razor blade from growth point, and the regenerated seedlings hypocotyls axis were whittled at sides into V-shape and sandwiched between the rootstock seedlings. The seedlings were twined with fine strings spliced from nylon grass and then placed in a plastic bag and sealed. The seedlings were cultured under the appropriate light and temperature, generally for about 10 days, to heal the wound.

(6) Exercise of the seedlings: The plastic bag was opened and stood for 7-10 days. The seedlings to be grafted were transplanted when new leaves grew.

(7) The plants were strictly inbred after blooming.

(8) The plants able to be used to receive seeds were subjected to molecule detection: firstly, 1500 mg/L Kanamycin was smeared on the main leaves of resistant plants, and non-transformed plants were used as control. Cotton total DNA was used as a template for molecular detection by PCR method with the following primers:

(SEQ ID No: 7)
5′ AGTCGTGGATGCGGGTTTGTTA 3′
(SEQ ID No: 8)
5′ GCAAGGCGATTAAGTTGGGTAA 3′

The reaction procedure was following: 95° C., 5 min; 95° C. 40 s, 58° C. 40 s, 72° C. 45 s, 32 cycles; 72° C. 10 min; 4° C., and then electrophoresis detection. The PCR amplification product at about 500 bp was the positive result.

(9) PCR positive plants were subjected to inbreed, so as to harvest inbred boll seeds. The inbred seeds were planted to the transgenic intermediate test base of the Cotton Research Institute of the Chinese Academy of Agricultural Sciences (the subject material was the control).

Totally 31 transgenic regeneration plants were obtained, and 31 plants were successfully grafted. PCR detection confirmed totally 18 positive plants. Each of the 18 plants gained more than 50 mature seeds.

### Example 4

### Southern Blotting of the Transgenic csRRM2 Gene Cotton Materials

The southern blotting of the transgenic csRRM2 gene cotton materials was carried out according to the following method:

(1) Total DNA from T0 generation transgenic cottons obtained in Example 3 was extracted in accordance with conventional methods.

(2) Digestion: Single enzyme digestion was carried out with restriction enzyme HindIII and EcoR I (purchased from the takara company) respectively at 37° C. for 6 hours. The digestion system was as follows: 30 μg DNA, 5.0 μl 10× digestion buffer, 5 μl restriction enzyme, adjusted with sterile deionized water to 50 μl. Digested samples were run on a 0.8% agarose gel electrophoresis, wherein the voltage was generally constant pressure: 1˜2 V/cm, the electrophoresis buffer was 0.5×TBE, and electrophoresis was stopped upon the bromophenol blue indicator was close (approximately 1 cm) to the other end of the gel.

(3) Labeling the probe: lug template DNA (plasmid pBIN438-csRRM2 with Bn-csRRM2 gene as the template is amplified by PCR, the primers are as follows:

(SEQ ID NO: 7)
5′ AGTCGTGGATGCGGGTTTGTTA 3′
(SEQ ID NO: 8)
5′ GCAAGGCGATTAAGTTGGGTAA 3′

The product was diluted with sterile water to 16 μl and denatured in boiling water for 10 min. DNA primers were immediately placed and mixed in the ice-water mixture (using DIG High Primer DNA Labeling and Detection Starter Kit I which was purchased from clone tech Company), 4 μl of which was then added into denatured DNA, mixed, inching centrifuged. After incubation at 37° C. overnight (20 h), 2 ul 0.2M EDTA was added to terminate the reaction, or heated at 65° C. for 10 min.

(4) DNA was transferred to a membrane, fixed, hybridized and the membrane was washed.

(5) ELISA detection: After hybridization and elution under stringent condition, the membrane was washed in washing buffer for 1-5 min, incubated in 100 ml blocking solution for 30 min, incubated in 20 ml antibody solution for 30 min, washed in 100 ml washing buffer twice, each for 15 min; balanced in 20 ml detecting buffer for 2-5 min, incubated in 10 ml new formulated color substrate solution in darkness, and the process completed after 16 h.

(6) Observation: After finishing development, hybrid membrane was taken out and developing solution was discarded. The membrane was flatly placed in TE buffer, and taken photographs by using a digital camera (FUJIFILM FinePix S8100fd, focal length: 10 mm) under natural light to save results.

The detection result of the Southern blot showed that 12 of the 18 positive plants obtained in Example 3 comprised at least one hybrid band, which indicated 12 transformants were obtained. The 12 transformants were numbered in order as L001, L002˜L012.

### Example 5

### Field Trials and Agronomic Traits Observation Test of the Bn-csRRM2 Gene Transgenic Materials

The trials and test were carried out in accordance with the following method:

(1) The 18 positive single plants (T0 generation) obtained in Example 3 were subjected to field trials using non-transgenic subject material CCRI 12 as control.

(2) All materials' agronomic traits (mainly plant height, leaf size, leaf color, bolling, disease resistance, boll opening etc.) were investigated in bud stage, the blooming period, boll period, boll opening stage of T1 generation materials (progeny of inbred T0 generation).

(3) Southern Blotting detection was performed on all materials, and the detection method was the same as in Example 4.

(4) The materials with a positive test result were numbered, respectively from L001 to L012 (the numbering was the same as above, the progeny of every plant material was numbered and named following this numbering). After blooming and then inbreeding, T2 generation seeds were harvested upon maturation.

(5) In the boll opening stage, 50 bolls were harvested from each plant line. Single boll weight, seed index, lint percentage, lint index and the like were observed. Fiber quality was tested by HVI900 fiber tester.

TABLE 1
Fiber quality test results of Bn-csRRM2 gene transgenic T1-generation
cotton (2008)

| | Fiber | | | | |
|---|---|---|---|---|---|
| Material | length | Uniformity | Micronaire | Elongation | Strength |
| number | (mm) | index (%) | value | (%) | (cN/tex) |
| L001 | 30.14 | 84.8 | 3.47 | 6.8 | 32.3 |
| L002 | 32.00 | 87.3 | 4.72 | 6.9 | 29.7 |
| L003 | 32.27 | 87.3 | 3.83 | 6.9 | 31.9 |
| L004 | 31.17 | 85.7 | 4.27 | 6.7 | 30.1 |
| L005 | 31.43 | 85.6 | 4.46 | 6.6 | 28.6 |
| L006 | 31.19 | 85.3 | 3.38 | 6.7 | 29.7 |
| L007 | 30.72 | 86.2 | 4.45 | 6.6 | 28.7 |
| L008 | 31.01 | 85.9 | 4.58 | 6.9 | 29.8 |
| L009 | 30.68 | 84.2 | 4.12 | 6.5 | 28.2 |
| L010 | 31.47 | 86.3 | 3.97 | 6.6 | 31.1 |
| L011 | 30.66 | 86.3 | 5.08 | 6.6 | 28.5 |
| L012 | 30.18 | 85.6 | 4.86 | 6.6 | 29.2 |
| average of | 31.08 | 85.9 | 4.27 | 6.7 | 29.8 |
| transgenic | | | | | |
| materials | | | | | |
| CCRI 12 | 27.84 | 84.5 | 4.42 | 6.2 | 26.0 |
| (CK) | | | | | |

The results (see Table 1): in the T1 generation, the average fiber length of transgenic materials increased by 3.24 mm as compared with the control, strength also improved 3.8 cN/tex. Other indexes were not significantly different from the control. These indicate that the Bn-csRRM2 gene can be used to improve cotton fiber quality.

### Example 6

### Field and Indoor Detection Test of T2-T5 Generation of Bn-csRRM2 Transgenic Cotton Material

(1) Material source: T2 generation was from the inbreeding of positive plants of T1 generation in Example 5 with excellent traits. Transgenic material of T3 generation was from the inbreeding of positive plants of T2 generation with excellent traits; transgenic material of T4 generation was from the inbreeding of positive plants of T3 generation with excellent traits; transgenic material of T5 generation was from the inbreeding of positive plants of T4 generation with excellent traits; controls were non-transgenic subject material CCRI 12.

(2) Cultivation methods: From 2008 to 2010, respectively, csRRM2 transgenic cotton materials of T2 T5 generations were subjected for field trials in the transgene intermediate test base of Cotton Research Institute of the Chinese Academy of Agricultural Sciences. 3 to 5 single plants of each T2 generation were planted. Planting in field was performed in random rows. The non-transgenic subject material CCRI 12 was the control.

(3) Block random test was carried out on T3 generation materials, repeating 2-3 times. Block settings were as follows: line length 6 m, line space 0.8 m, 3 lines; planting T4 generation T5 generation materials in the same way with T3 generation materials; non-transgenic subject material CCRI 12 is the control.

(4) Field observation and test: field observation and indoor species test were carried out on traits including cotton cotyledons, bracts, leaves, petioles, flowers, cotton bolls, ovaries boll sections, boll husk and cotton fiber. The plants detected of positive and with superior performance were used to inbreed into the next generation.

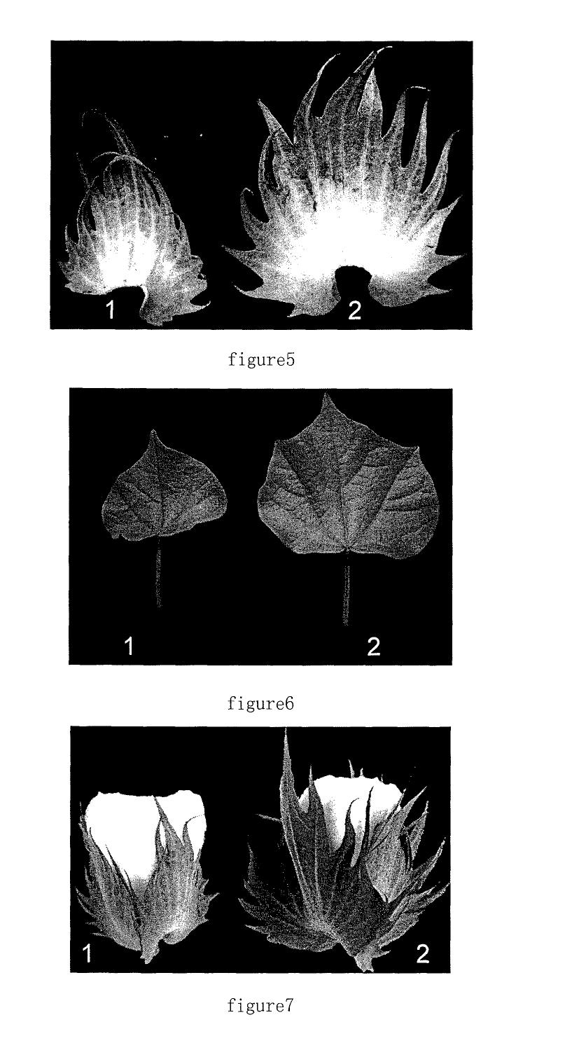

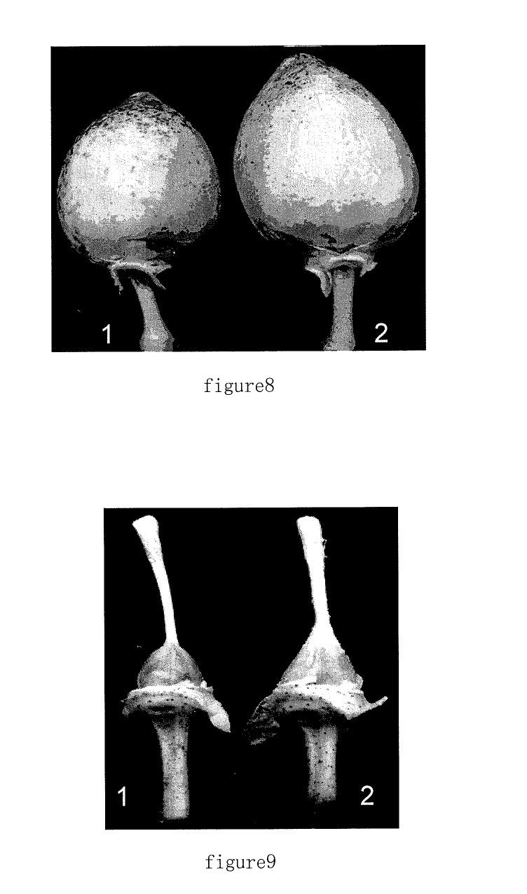

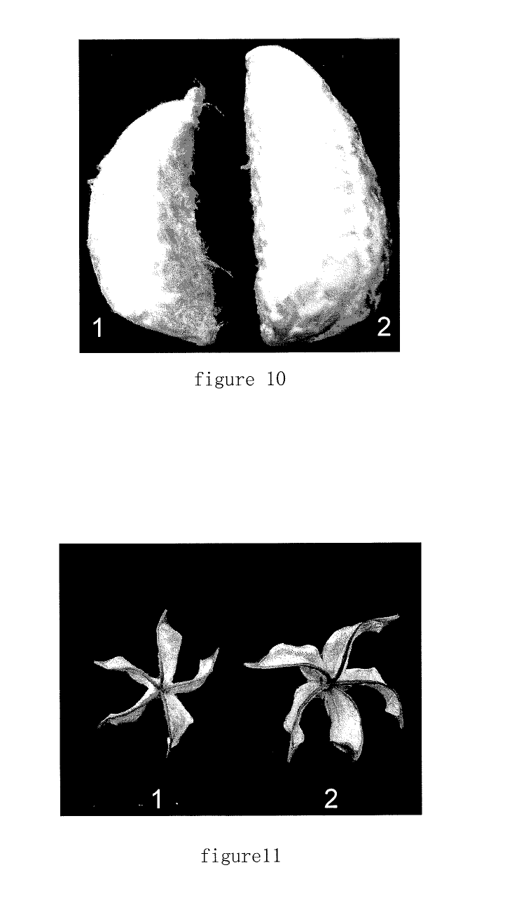

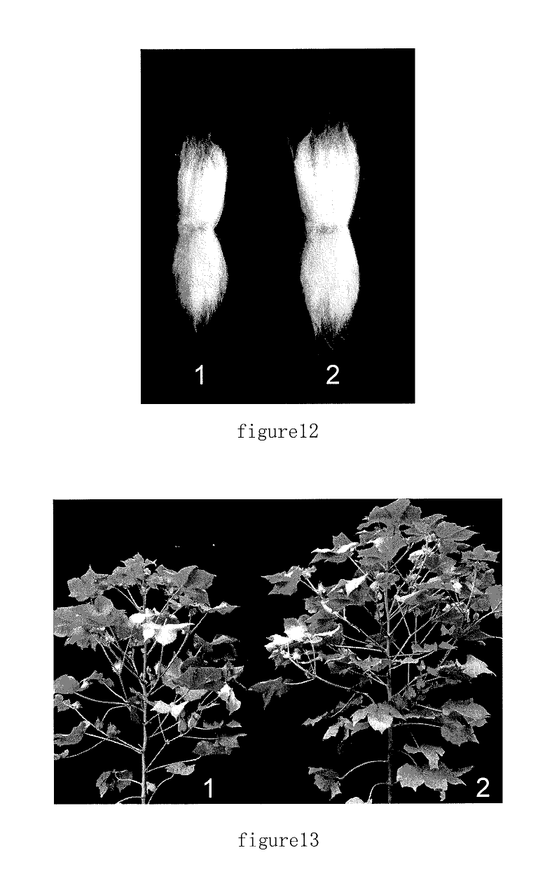

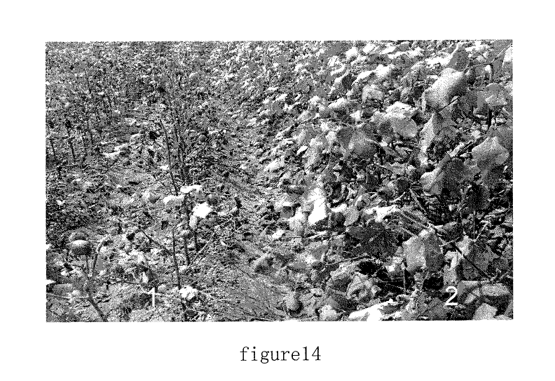

Results: (a) in all generations (see FIGS. 5 to 12), traits of transgenic material including the cotyledons, bracts, leaves, petioles, flowers, cotton bolls, ovary, boll husks, boll sections, cotton fibers increased significantly. Stems of transgenic regenerated plants became thicker; the plants became larger (see FIG. 13). Bolls of transgenic materials (see FIG. 8) were significantly larger than the subject materials.

(b) Single boll weight and boll: data of T2, T3 generation materials planted in the field (investigated in 2009) are shown in Table 2 and Table 3 (data after T3 generation is basically the same, and thus omitted here).

TABLE 2
Testing results of the BN-csRRM2 transgenic cotton materials of T2
generation

| | Single boll | Single plant | Seed | lint percentage |
|---|---|---|---|---|
| Tested material | weight (g) | bolls (No.) | index (g) | (%) |
| CCRI 12 (CK) | 5.23 | 14.8 | 9.43 | 40.40 |
| L001 | 6.67 | 12.6 | 12.70 | 37.14 |
| L002 | 6.04 | 17.8 | 10.87 | 39.27 |
| L003 | 6.13 | 18.6 | 10.64 | 35.63 |
| L004 | 5.82 | 15.2 | 12.20 | 38.83 |
| L005 | 5.68 | 18.4 | 10.20 | 42.47 |
| L006 | 5.49 | 13.4 | 9.90 | 37.16 |
| L007 | 6.65 | 17.6 | 11.36 | 36.65 |
| L008 | 6.80 | 16.8 | 10.75 | 37.67 |

TABLE 3
Results of traits of the BN-csRRM2 transgenic cotton materials of T3
generation

| | Single boll | Single plant | Seed | lint percentage |
|---|---|---|---|---|
| Tested material | weight (g) | bolls (No.) | index (g) | (%) |
| CCRI 12 (CK) | 5.23 | 14.8 | 9.43 | 40.40 |
| L009 | 6.17 | 18.4 | 11.70 | 36.69 |
| L010 | 7.84 | 17.4 | 11.11 | 38.78 |
| L011 | 7.64 | 16.2 | 10.00 | 36.01 |
| L012 | 6.01 | 20.2 | 11.24 | 37.16 |
| Average | 6.915 | 18.05 | 11.013 | 37.16 |

The boll weight, bolls per plant, seed index of transgenic materials were significantly higher than the control. For instance, in T2 generation, transgenic material L008 had single boll weight of 6.80 g, which was 30% higher than that in the control material; in the T3 generation, L010, L011 have single boll weight of 7.64 g and 7.84 g respectively, which increased by 46% and 50% compared with the control, respectively. With regard to boiling compared to the control, except L001 and L006 were slightly weaker, other materials were significantly stronger than the control, in which bolls per plant of L012 was 36% higher over the control.

These results suggest that Bn-csRRM2 gene can be used for the improvement of cotton yield and quality traits.

(c) Growth duration (see FIG. 14): Growth duration of all transgenic materials extended. In particular, to the late stage, leaves of control materials almost fell off, but transgenic materials were still lush foliage. The extension of growth duration helps to improve the yield and fiber quality.

(d) Fiber quality: The average of the detection results in T4, T5 generations are shown in Table 4. (Due to the results of T2, T3 generations were substantially the same, they are omitted here). In T4/T5 generation, the fiber quality of four independently transformed lines (9304-1 etc. Numbers represent different plants) significantly improved comparing with the control in fiber length and fiber strength. At the same time, the boll weight of transgenic materials also improved significantly compared with the control: the boll weight of two lines of L010 was still about 7.5 grams. The boll weight of all lines increased by more than 27% compared with the control. These results show the improvement in fiber quality can be achieved synchronously with the improvement in boll weight.

The above results illustrate the single boll weight and bolling of Bn-csRRM2 transgenic cotton materials were enhanced; the fiber quality of transgenic materials significantly improved; transgenic materials became significantly larger at organ level; the increase of single boll weight and the improvement of fiber quality can be achieved synchronously.

After five generations of field trials, 9 transgenic lines with significant boll weight increase and fiber quality improvement were selected, respectively 9317-1, 9317-2 and 9316-2 from L011; 9304-1, 9304-3 from L010; 9311-3, 9311-5 from L003; 9319-1, 9319-4 from L004.

TABLE 4
Test results of lines of Bn-csRRM2 transgenic cotton from T4/T5 generation (2010)

| | | | | Fiber quality | | | |
|---|---|---|---|---|---|---|---|
| Test | | | Single boll | Length | Micronaire | Elongation | Strength |
| materials | No. | Generation | weight | (mm) | value | rate (%) | (cN/tex) |
| L010 | 9304-1 | T5 | 7.70 | 31.50 | 5.28 | 6.9 | 31.16 |
| L010 | 9304-3 | T5 | 7.40 | 31.85 | 5.05 | 8.4 | 29.50 |
| L011 | 9316-2 | T5 | 6.38 | 32.43 | 3.53 | 7.2 | 31.36 |
| L011 | 9317-1 | T5 | 7.86 | 33.21 | 4.09 | 6.1 | 34.12 |
| L011 | 9317-2 | T5 | 7.52 | 32.18 | 4.19 | 6.1 | 33.52 |
| — | Average | — | 7.25 | 31.99 | 4.51 | 7.1 | 31.39 |
| L003 | 9311-3 | T4 | 6.75 | 30.22 | 3.95 | 6.1 | 32.93 |
| L003 | 9311-5 | T4 | 6.69 | 30.86 | 4.43 | 7.9 | 34.59 |
| L004 | 9319-1 | T4 | 6.58 | 29.50 | 4.69 | 7.1 | 33.22 |
| L004 | 9319-4 | T4 | 6.82 | 30.00 | 4.60 | 8.2 | 31.36 |
| — | Average | — | 6.71 | 30.15 | 4.42 | 7.3 | 33.03 |
| CK | CCRI12 | | 5.18 | 28.88 | 3.54 | 7.1 | 28.11 |

### Example 6

### Comparative Test of Hybrids Bred from Bn-csRRM2 Transgenic Cotton as the Parent Group

Comparative test was carried out in accordance with the following method:

(1) Hybrid combination set up: two transgenic lines 9422 (T3 generation from L010) and 8350 (T3 generation from L003) were selected to plant in field in blocks (line length 5 m, line space 0.9 m, 2 lines). Line 9422 or 8350 was used as the female parent to hybrid with Zhong287 (large bell materials from Cotton Research Institute of the Chinese Academy of Agricultural Sciences) and Zhong3316 (small bell materials from Cotton Research Institute of the Chinese Academy of Agricultural Sciences) hybridization. The formulated combinations were shown in Table 6.

(2) Hybrid combination of materials prepared (1) were planted, with its parents as the control, CCRI 12 as negative control. Randomized Blocks were designed randomly, and each block was designed as follows: 3 lines, line length 7 m, line space 0.8 m.

(3) Growth duration, single boll weight, fiber quality traits were tested the same as in Example 5.

Results: (a) the appearance of hybrid materials was substantially the same as the parents, and the characteristic of enlarged organ was maintained.

(b) The boll weight of hybrid materials (see Table 5) was not significantly different from the female parent, but significantly larger than the male parent. Regarding the bolling of hybrid materials, two combinations (8350 X Zhong3316, 9422 X Zhong287) were significantly higher, and the other two combinations were not significantly different from the parents.

(c) The fiber quality of the hybrid materials (Table 5) was similar with or stronger than the female parent, but significantly improved compared with the parent. For instance, 9422 X Zhong287 had a fiber length of 30.69 mm and a breaking strength of 33.1 cN/tex, which were essentially equal to the female parent (fiber length of 30.73 mm, breaking strength of 32.3 cN/tex). The fiber strength of 9422 X Zhong3316 was significantly stronger than that of the parent.

These results suggest that the Bn-csRRM2 gene can increase the yield and improve the fiber quality of cotton.

TABLE 5
Comparison of experimental results of Bn-csRRM2 transgenic cotton hybrids

| | Yield traits | | Fiber quality traits | | | | |
|---|---|---|---|---|---|---|---|
| Test | Single boll | Bolls | Length | Uniformity | Micronaire | Elongation | Strength |
| materials | weight (g) | number | (mm) | (%) | value | rate (%) | (cN/tex) |
| 9422 | 7.40 | 18.3 | 30.69 | 86.3 | 4.05 | 6.8 | 33.1 |
| X | | | | | | | |
| Zhong287 | | | | | | | |
| 9422 | 7.70 | 17.3 | 31.53 | 86.7 | 3.67 | 6.8 | 36.6 |
| X | | | | | | | |
| Zhong3316 | | | | | | | |
| 8350 | 6.85 | 18.4 | 29.46 | 88.4 | 4.09 | 6.8 | 31.4 |
| X | | | | | | | |
| Zhong287 | | | | | | | |
| 8350 | 6.43 | 19.2 | 27.37 | 87.1 | 4.22 | 6.8 | 28.8 |
| X | | | | | | | |
| Zhong3316 | | | | | | | |
| 9422 | 7.52 | 15.2 | 30.73 | 86.3 | 4.08 | 6.80 | 32.3 |
| 8350 | 6.76 | 16.6 | 28.11 | 85.9 | 4.38 | 6.80 | 27.7 |
| Zhong287 | 5.64 | 17.2 | 27.50 | 83.3 | 4.40 | 5.2 | 25.7 |
| Zhong3316 | 4.11 | 16.6 | 28.21 | 86.2 | 3.76 | 6.4 | 28.5 |
| CCRI12 | 5.01 | 16.5 | 26.92 | 86.8 | 3.41 | 6.7 | 29.3 |

1. A protein associated with crop yield or quality traits, characterized in that it is the protein as shown in the following (a) or (b):
(a) A protein consisting of the amino acid sequence of SEQ ID No: 1;
(b) A protein consisting of the amino acid sequence of SEQ ID No: 1 with deletion, addition or substitution of one or more amino acid residues, and associated with crop yield or quality traits.

2. The protein according to claim 1, characterized in that the crop is cotton (Gossypium hirusute L.), maize (Zea mays L.), rice (Oryza sativa L.), wheat (Triticum aestivum L.), barley (Hordeum L.), or rape (Brassica napus L.).

3. A gene encoding the protein according to claim 1, which is shown as the following 1) or 2) or 3):
1) A gene consisting of the nucleotide sequence of SEQ ID No: 2;
2) A gene consisting of the nucleotide sequence of SEQ ID No: 2 with mutation, substitution or deletion of one or more bases, and encoding the protein associated with crop yield or quality traits;
3) A gene consisting of a nucleotide sequence which can hybridize with the DNA sequence defined by SEQ ID No.: 2 under stringent conditions and encoding the protein associated with crop yield or quality traits.

4. An expression vector comprising the encoding gene of claim 3.

5. A transgenic plant cell line comprising the encoding gene of claim 3.

6. A transgenic plant tissue comprising the encoding gene of claim 3.

7. A transgenic plant comprising the encoding gene of claim 3.

8. A method for cultivating plant varieties, characterized in that using the transgenic plant of claim 7 as one of the parents, and cultivating plant varieties with improved yield or quality traits by backcross or hybrid methods.

9. A hybrid breeding method, characterized in that using the transgenic plant of claim 7 or 8 as one of the parents to obtain the hybrid plant.

10. A PCR kit for detecting the encoding gene of claim 3, characterized in that the primers used for PCR amplification are:
(SEQ ID NO. 7)
5′ AGTCGTGGATGCGGGTTTGTTA 3′,
and
(SEQ ID NO. 8)
5′ GCAAGGCGATTAAGTTGGGTAA 3′.


SEQUENCE LISTING (8 sequences)

SEQ ID NO: 1 - protein, 77 aa, Brassica napus
KLFVGSLNKQATENEVEELFLQFGRVEDVYLMRDEYRQSRGCGFVKYSSKETAMAAIDGLNGTYTMRGCNQPLIVRF

SEQ ID NO: 2 - DNA, 231 bp, Brassica napus
aagctttttgttggttccttaaacaagcaagccactgaaaacgaggttgaggagctctttttgcaatttggtcgcgtggaggatgtctatctcatgcgtgatgaatatagacagagtcgtggatgcgggtttgttaaatattcaagcaaagagacggccatggcagctatcgatggtctcaatggaacttataccatgagaggttgcaatcagccgttgattgttcggttt

SEQ ID NO: 3 - DNA, 25 bp, Artificial
  Bn-csRRM2-S
gaggatccatgggtgcggtagagtt

SEQ ID NO: 4 - DNA, 25 bp, Artificial
  Bn-csRRM2-A
cgtagatcttgtgccacttcccttg

SEQ ID NO: 5 - DNA, 328 bp, Artificial
  ORF of Bn-caRRM2 in expression vector
atgggtgcggtagagtttaagctttttgttggttccttaaacaagcaagccactgaaaacgaggttgaggagctctttttgcaatttggtcgcgtggaggatgtctatctcatgcgtgatgaatatagacagagtcgtggatgcgggtttgttaaatattcaagcaaagagacggccatggcagctatcgatggtctcaatggaacttataccatgagaggttgcaatcagccgttgattgttcggtttgctgatccaaagaggcctaaaccgggggagtcaagggaagtggcacatctagaggatcgatccccgcttaattaattaa

SEQ ID NO: 6 - protein, 107 aa, Artificial
  amino acid sequence in expression vector
MGAVEFKLFVGSLNKQATENEVEELFLQFGRVEDVYLMRDEYRQSRGCGFVKYSSKETAMAAIDGLNGTYTMRGCNQPLIVRFADPKRPKPGESREVAHLEDRSPLN

SEQ ID NO: 7 - DNA, 22 bp, Artificial
  detection primer 1
agtcgtggatgcgggtttgtta

SEQ ID NO: 8 - DNA, 22 bp, Artificial
  detection primer 2
gcaaggcgattaagttgggtaa